(19) United States
(12) Patent Application Publication
(10) Pub. No.: US 2026/0216009 A1
(43) Pub. Date: Jul. 30, 2026

(54) Microneedle Particles and Methods of Incorporating a Substance Into or Onto Microneedle Particles

(71) Applicant: Georgia Tech Research Corporation, Atlanta, GA (US)

(72) Inventors: Mark R. Prausnitz, Atlanta, GA (US); Andrew Tadros, Atlanta, GA (US)

(21) Appl. No.: 19/143,555

(22) PCT Filed: Jan. 4, 2024
(86) PCT No.: PCT/US2024/010338

§ 371 Date: Jun. 26, 2025

Related U.S. Application Data
(60) Provisional application No. 63/436,998, filed on Jan. 4, 2023.

(51) Int. Cl. A61K 8/02; A01N 25/12; A01P 17/00; A61Q 17/04

(52) U.S. Cl. CPC A61K 8/0245; A01N 25/12; A01P 17/00; A61K 8/0279; A61Q 17/04

(57) ABSTRACT

STAR particles, compositions, and methods of making the same, where the STAR particles are configured to mechanically disrupt a biological tissue surface. The STAR particles may be effective to administer a substance of interest (SOI) to, or remove a SOI from, a biological tissue such as a patient's skin. The composition may include a plurality of STAR particles which are configured to mechanically disrupt a biological tissue surface, wherein the STAR particles have a heterogeneous structure comprising (i) a first SOI contained within a shell of carrier material, (ii) one or more layers of a first SOI stacked with one or more layers of a carrier material; or (iii) dispersed domains containing a first SOI, which optionally consists of a suspension of solid SOI-containing particles, dispersed in a solid medium of carrier material.

### CROSS-REFERENCE TO RELATED APPLICATIONS

This application claims priority to U.S. Provisional Application No. 63/436,998, filed Jan. 4, 2023, which is incorporated herein by reference.

### BACKGROUND

Some embodiments of microneedle particles (i.e., STAR particles) are described in U.S. Pat. No. 11,291,816, which is incorporated herein by reference. STAR particles may be effective to treat biological barriers and enhance topical delivery of compounds to target tissues, specifically for enhancing topical delivery of compounds to the skin. It would be desirable to provide alternative or improved means for controlling the delivery or dosage of various substances of interest with the use of STAR particles.

### BRIEF SUMMARY

In one aspect, a composition having a plurality of STAR particles is provided, where the STAR particles are configured to mechanically disrupt a biological tissue surface. The STAR particles may have, at least in part, a porous structure. For example, the STAR particles may have pores comprising a first substance of interest (SOI) in (i) liquid or gaseous form contained in the pores, or (ii) solid form as a coating on surfaces defining the pores. The pores may include the first SOI in the liquid form, or may include the first SOI in the solid form as a coating on surfaces defining the pores, optionally where the first SOI completely or substantially completely fills the pores. The composition may also include a vehicle in which the plurality of STAR particles are dispersed. The vehicle may include the first SOI and/or a second SOI, or the vehicle may include no or substantially no SOI. The first and/or second SOI may be a bioactive agent.

In another aspect, a composition having a plurality of STAR particles is provided, where the STAR particles are configured to mechanically disrupt a biological tissue surface. The STAR particles may include a gas, where the gas is adsorbed or absorbed onto or into the STAR particles.

In a further aspect, a composition having a plurality of STAR particles is provided, where the STAR particles are configured to mechanically disrupt a biological tissue surface. The STAR particles have a heterogeneous structure comprising (i) a first substance of interest (SOI) contained within a shell of carrier material, (ii) one or more layers of a first SOI stacked with one or more layers of a carrier material; or (iii) dispersed domains containing a first SOI, which may consist of a suspension of solid SOI-containing particles, dispersed in a solid medium of carrier material. The carrier material may include a pharmaceutically acceptable excipient. The composition may also include a liquid vehicle in which the plurality of STAR particles are dispersed. The liquid vehicle may include a second SOI. The first and/or the second SOI may include a bioactive agent.

In some more particular embodiments, the STAR particles are planar particles. In some embodiments, the STAR particles are configured to mechanically disrupt the stratum corneum of a patient's skin.

In yet another aspect, a method of making STAR particles is provided, the method including the steps of dissolving or dispersing a substance of interest (SOI) into a carrier material to form a fluid composition, and casting the fluid composition in molds to form the STAR particles, where the fluid composition comprises (i) a single phase, or (ii) two or more phases, such as a suspension.

In an even further aspect, a method of making STAR particles is provided, the method including the steps of forming a plurality of porous STAR particles, and then contacting the formed porous STAR particles with a liquid, gas, solid, or any combination thereof, containing a substance of interest (SOI) in a manner such that the liquid, gas, solid, or a combination of two or three phases thereof is incorporated into pores of the STAR particles. In some embodiments, the method also includes converting the SOI of the liquid incorporated into pores of the STAR particles into a solid or gaseous form, or converting the SOI of the gas incorporated into pores of the STAR particles into a solid or liquid form.

In another aspect, a method of making STAR particles is provided, the method including the steps of forming a plurality of STAR particles, and then adsorbing, absorbing, or spraying a solid form of a substance of interest (SOI) into/onto the STAR particles. In some embodiments, the STAR particles are porous.

In an even further aspect, a method of making STAR particles is provided, the method including the steps of providing a film which comprises a substance of interest (SOI) and a carrier material, and cutting one or more STAR particles out from the film. The cutting may include stamping or laser cutting. In some embodiments, the carrier material includes one or more pharmaceutically acceptable excipients.

In more particular embodiments, the SOI includes a bioactive agent. In some embodiments, the STAR particles are planar particles. In some embodiments, the STAR particles are configured to mechanically disrupt the stratum corneum of a patient's skin.

In a further aspect, a method of transferring a biological substance from within a patient's skin to a surface of a patient's skin and/or into/onto a STAR particle is provided, the method including the steps of contacting the skin with a plurality of STAR particles in a manner to create one or more holes in the stratum corneum of the patient's skin effective to cause the biological substance within the skin to be transferred onto the skin surface and/or into/onto the STAR particles. The biological substance may collected from the skin surface and/or from the STAR particles for diagnostic and/or monitoring purposes. In some embodiments, the STAR particles are configured to selectively uptake the biological substance. In some embodiments, the biological substance on the skin surface combines with a vehicle of a composition containing the STAR particles applied to the skin. The vehicle may be configured to selectively uptake the biological substance. In some embodiments, the biological substance is interstitial fluid or a biomarker.

In some embodiments, the method further includes applying pressure to the skin surface to withdraw the biological substance from the skin. In some embodiments, the pressure is a positive pressure, such as squeezing the skin surface. In other embodiments, the pressure is a negative pressure, such as applying suction to the skin surface.

In yet another aspect, a method of administering a substance of interest (SOI) to a patient's skin is provided, the method including the steps of contacting the patient's skin with a composition having a plurality of STAR particles and a vehicle in a manner to mechanically disrupt the stratum corneum of a patient's skin, transforming the composition to form a film on the patient's skin, and incorporating the SOI into the film and/or into an interface between the skin and the film. The transforming may include dissolution, melting, and/or plastic deformation of the STAR particles.

In an additional aspect, a method of administering a substance of interest (SOI) to a patient's skin is provided, the method including the steps of applying to the patient's skin a plurality of STAR particles, and rubbing the plurality of STAR particles in a manner effective to mechanically disrupt the stratum corneum of the patient's skin, where a SOI is released from the STAR particles during and/or after the rubbing. In some embodiments, the SOI is released from the STAR particles upon one or more tip portions of microneedles of the STAR particles penetrating into the skin and dissolving or breaking off therein or on the surface of the skin. In some other embodiments, the SOI is released from the STAR particles onto the surface of the skin but not substantially into the skin during the rubbing. In some other embodiments, after the rubbing, the SOI is absorbed into the skin.

In a further aspect, a method is provided, the method including the steps of applying to a patient's skin a plurality of STAR particles comprising a substance of interest (SOI), and rubbing the plurality of STAR particles against the skin, where no or substantially no SOI is released from the STAR particles during the rubbing. In some embodiments, the SOI is released from the STAR particles onto the skin after the rubbing and then is absorbed into the skin. In some other embodiments, the SOI is released from the STAR particles after the rubbing and is not substantially absorbed into the skin. In some embodiments, the SOI comprises a compound which absorbs UV radiation or otherwise is effective as a sunscreen. In some embodiments, the SOI comprises a compound configured to evaporate from the skin surface. In some embodiments, the SOI comprises a perfume or insect repellent.

In yet an additional embodiment, a composition is provided, the composition including a plurality of STAR particles which comprise a first substance of interest (SOI) which comprises (i) a compound which absorbs UV radiation or otherwise is effective as a sunscreen, (ii) a perfume and/or (iii) an insect repellent. In some embodiments, the STAR particles also include a carrier material. In some embodiments, the STAR particles are configured such that the first SOI is not released from the STAR particles following application of the composition to skin. In some embodiments, the composition also includes a liquid vehicle in which the STAR particles are dispersed. In some embodiments, the composition also incudes a second SOI on or in the STAR particles and/or in the liquid vehicle. The second SOI may be configured to be absorbed into skin following application to the composition to the skin. In some embodiments, the STAR particles are planar particles. In some embodiments, the STAR particles are configured to mechanically disrupt the stratum corneum of a patient's skin.

### BRIEF DESCRIPTION OF THE FIGURES

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components are not necessarily drawn to scale.

FIG. 1A is a plan view of a planar STAR particle according to one embodiment of the present disclosure.

FIG. 1B is a perspective view of the planar STAR particle of FIG. 1A.

FIG. 1C is a plan view of a planar STAR particle according to another embodiment of the present disclosure.

FIG. 1D is a perspective view of the microneedle particle of FIG. 1C.

FIG. 1E is a side view of the microneedle particle of FIG. 1C.

FIG. 2A is a plan view of a planar STAR particle according to another embodiment of the present disclosure, in which the STAR particle is fully coated in a substance of interest (SOI).

FIG. 2B is a plan view of a planar STAR particle according to another embodiment of the present disclosure, in which the STAR particle is partially coated in a SOI.

FIG. 2C is a plan view of a planar STAR particle according to another embodiment of the present disclosure, in which the STAR particle partially comprises a SOI.

FIG. 2D is a plan view of a planar STAR particle according to another embodiment of the present disclosure, in which the STAR particle is porous.

FIGS. 3A-3B depict a method, according to an embodiment of the present disclosure, that includes pretreating tissue with a STAR particle, and delivering a SOI into the pretreated tissue with a STAR particle.

FIG. 4A depicts a method of delivery a SOI into tissue from a STAR particle, according to another embodiment of the present disclosure.

FIG. 4B depicts a method of delivery a SOI onto tissue from a STAR particle, according to another embodiment of the present disclosure.

FIG. 5A depicts a method of delivering a SOI to the skin, where the SOI is already present on the skin, according to another embodiment of the present disclosure.

FIG. 5B depicts a method of delivering a SOI onto the skin, where the SOI is already present on the skin, according to another embodiment of the present disclosure.

FIG. 6A depicts a method of removing a biological specimen from the skin, according to an embodiment of the present disclosure.

FIG. 6B depicts a method of reintroducing a biological specimen into the skin, according to another embodiment of the present disclosure.

FIG. 6C depicts a method of reintroducing a biological specimen onto the skin, according to another embodiment of the present disclosure.

FIG. 6D depicts a method of introducing a biological specimen to a formulation or to the environment surrounding the tissue, according to another embodiment of the present disclosure.

### DETAILED DESCRIPTION

STAR particles, methods of synthesis, and methods of use are disclosed, including alternative or improved means for controlling the delivery or dosage of various substances of interest using STAR particle-containing compositions. For example, this may include modifying the spatial or temporal delivery profile and/or increasing or decreasing a dosage of a bioactive agent or other substance.

The STAR particle-containing compositions and methods disclosed herein may enhance topical delivery of bioactive agents and other substances of interest to improve the desired effects of the compounds, to facilitate the compounds remaining in and/or on the target tissue, to facilitate extraction or removal of endogenous substances, compounds and/or specimens from a target tissue, and/or to promote systemic uptake of the compounds. The STAR particle-containing compositions and methods disclosed herein may be useful for diagnostic, prognostic, therapeutic, adjuvant, cosmetic and/or preventative purposes.

STAR particles may enhance topical administration of another substance or substances by mechanically disrupting the integrity of an outer/upper layer of skin (or other biological tissue) to facilitate the substance(s) local delivery into/onto the target tissue of a patient, and/or to promote its passage through a target tissue and uptake in the bloodstream for systemic delivery and/or lymphatics for systemic delivery, and/or to promote the substance(s)' passage through a target tissue and uptake into another tissue or space including, but not limited to, joint spaces, tendons, ligaments, fascia, nerves, vessels, bones, muscles, glands, lymph nodes, subcutaneous tissue, adipose tissue, organs, and/or other tissues and spaces. The patient may be a human or other mammal or other animal or plant. The skin, or other biological tissue, may be in vivo or ex vivo.

As used herein, a STAR particles being configured to “mechanically disrupt” a biologically tissue, particularly the stratum corneum of mammalian skin, particularly, human skin refers to the particle having dimensions and mechanical strength capable of creating holes or pores in the tissue surface. For example, the mechanically disrupting may be forming a penetration through the stratum corneum.

In some embodiments, the STAR particles may include a SOI that is not intended to be absorbed into the skin of a person onto which the STAR particles have been applied. The SOI may instead provide a useful function on the skin's surface or in the air or environment near the person. Non-limiting examples include sunscreens, perfumes, and insect repellants.

Compositions of such STAR particles may, however, further include one or more additional substances of interest that are intended to be absorbed into the skin following mechanical disruption of the stratum corneum using the STAR particles of the composition.

### STAR Particles

The STAR particles include a core structure and one or more microneedle-like projections extending from the core structure. The microneedles may be structured to at least partially penetrate or otherwise mechanically disrupt a biological tissue, such as the stratum corneum of human skin (or other biological tissue). That is, the microneedles are dimensioned and possess the mechanical rigidity to enable them to be pressed into and penetrate the biological tissue, forming a microscale hole or channel therein. The microneedles may extend independently in any direction from the core structure.

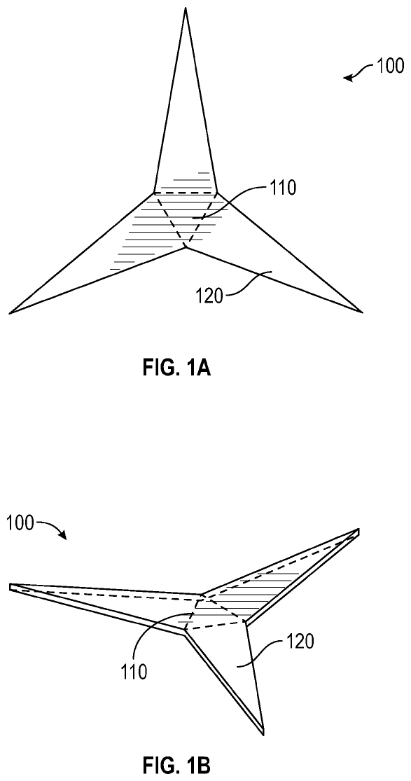

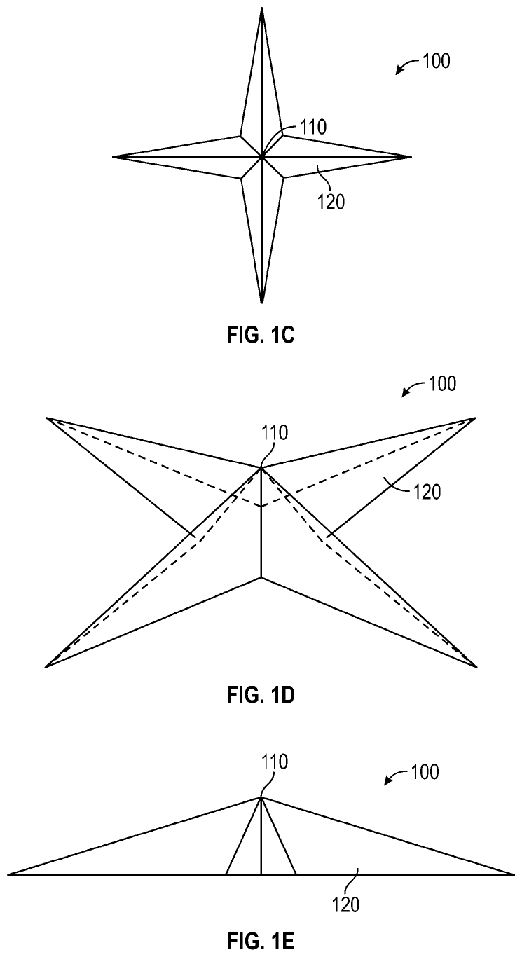

FIGS. 1A and 1B depict a STAR particle 100 according to one embodiment. In this embodiment, the STAR particles 100 each have three microneedles 120 extending from the core structure 110 in the same plane, such that the STAR particle is referred to as a planar particle. The core structure typically is the portion of the microneedle particle that connects the microneedles, especially when there are multiple microneedles. The core structure may be a solid structure, a porous structure, or a hollow structure having one or more internal cavities. In other embodiments, the STAR particles may have two microneedles, four microneedles, five microneedles, six microneedles, seven microneedles, eight microneedles, nine microneedles, or ten microneedles extending from the core structure. In some embodiments, the microneedles extend from the core in different planes. For example, the STAR particle may have three, four, five, or more microneedles extending in different directions and planes, such that the STAR particle is referred to as a non-planar particle.

The microneedles of STAR particles may be tapered. In some embodiments, in the plan view, as shown in FIG. 1A, the microneedle 120 tapers from the core structure 110 to the tip end, but the height of the microneedles is substantially constant. In some other embodiments. the edges of the microneedle 120 may also be tapered, as the tapered edges are sharp and thereby able to penetrate stratum corneum more easily than untapered edges. For example, in some embodiments, as shown in FIGS. 1C-1E, the microneedles 120 may taper both in width and in height. That is, the height of the microneedle is largest at the core structure and smallest at the tip. In other variations, the core and a base portion of the microneedles may have a uniform height and only the distal tip portion of the microneedles is tapered. In variations, the taper may be from one or both sides of the STAR particle.

Various design features of the STAR particles may be selected to impart the particles with the functionality preventing the entire STAR particle from penetrating (i.e., mechanically disrupting) a biological tissue. These features may include the core structure itself, the microneedles themselves, or the spatial relationship between/among the microneedles or a subset of those microneedles. A combination of these features may be designed to prevent the entire STAR particle from penetrating a biological tissue.

For example, the core structure may have a size, shape, and/or a lack of sharp edges that permits one or more of the microneedles extending from the core structure to penetrate a biological tissue, but that inhibits all or substantially all of the core structure from penetrating into the biological tissue. As a further example, the microneedles may have a structural feature, such as tapering, that permits only a portion (i.e., the tip portion distal to the core structure) of the microneedles to penetrate a biological tissue. For example, a microneedle may have a shoulder or plateau that permits only the portion of the microneedle distal to the shoulder or plateau to penetrate the biological tissue. Such a configuration may prevent the core structure from contacting the biological tissue. In another example, the microneedle particle may have a generally curved shape that comes to a tip and, due to the geometry, only permits the tip portion to penetrate the biological tissue.

Generally, the microneedles of a STAR particle can have the same or different dimensions and/or geometries from one another. In one embodiment, the microneedles of a planar STAR particle have substantially the same dimensions and geometries.

The microneedles may have any shape effective to at least partially penetrate a biological tissue. In some embodiments, the microneedles are high-aspect-ratio structures having a length at least two times greater than its width at the base of the microneedle (i.e., at the interface of the microneedle and core structure). The length of a microneedle is the distance from the interface of the microneedle and the core structure's edge to the tip of the microneedle. In some embodiments, each of the microneedles independently has a length from 1 μm to 2,000 μm. In some embodiments, each of the microneedles independently has a length from 10 μm to 2,000 μm. In some embodiments, each of the microneedles independently has a length from 50 μm to 2,000 μm. In some embodiments, each of the microneedles independently has a length from 100 μm to 1,000 μm. In some embodiments, each of the microneedles independently has a length from 250 μm to 750 μm. In some embodiments, each of the microneedles independently has a length from 100 μm to 500 μm. In some embodiments, each of the microneedles has a length of about 350 μm.

In a particular embodiment, the STAR particles have three microneedles, wherein each of the microneedles independently has a length of about 1 μm to about 2,000 μm, about 10 μm to about 2,000 μm, about 50 μm to about 2,000 μm, about 100 μm to about 1,000 μm, or about 250 μm to about 750 μm. This STAR particle may be a planar particle.

The microneedles of the STAR particle may have a tip having a radius of curvature of about 0.1 μm to about 50 μm. In some embodiments, the microneedles have a tip having a radius of curvature of about 0.1 μm to about 50 μm, about 0.1 μm to about 25 μm, about 0.1 μm to about 20 μm, about 0.1 μm to about 15 μm, about 0.1 μm to about 10 μm, about 0.1 μm to about 5 μm, about 1 μm to about 10 μm, about 1 μm to about 7 μm, about 1 μm to about 5 μm, about 1 μm to about 4 μm, or about 1 μm to about 3 μm, about 5 μm to about 50 μm, about 5 μm to about 25 μm, about 5 μm to about 20 μm, about 5 μm to about 15 μm, or about 5 μm to about 10 μm. In some embodiments, each microneedle has a tip having a radius of curvature of about 5 μm to about 30 μm. The “tip” typically is the portion of the microneedles that first penetrates a biological tissue.

In some embodiments, the STAR particles are shaped and sized to prevent, or reduce the likelihood of, the STAR particle becoming completely or irremovably embedded in the biological tissue. In some embodiments, the greatest dimension of the STAR particles is about 100 μm to about 5,000 μm, 100 μm to about 10,000 μm, about 250 μm to about 5,000 μm, about 500 to about 2,000 μm, or about 500 μm 1,000 μm. The “greatest dimension of the STAR particles” refers to the greatest of the following distances: [1] the distance between the tips of the two microneedles that are the farthest apart (if the microneedle particle includes two or more microneedles), or [2] the farther possible distance between a tip of a microneedle and the side of the core structure that is opposite the side from which the measured microneedle extends. A plurality of microneedle particles may include microneedle particles of one or more sizes.

In some embodiments, the microneedles of a STAR particle are planar microneedles. The phrase “planar microneedles,” as used herein, refers to two or more microneedles, each having either [1] a central axis that extends from the core structure in at least substantially the same plane, or [2] a tip that exists in substantially the same plane. The planar microneedles may include microneedles that extend from the core structure in the same direction, different directions, or a combination thereof. The planar microneedles also may include co-linear planar microneedles, which extend from opposite sides of the core structure in a manner that permits the central axis of each microneedle to at least substantially correspond with a single line. For example, where the STAR particles include two or more pairs of microneedles, the pairs of microneedles, but not necessarily all microneedles, may be co-linear.

When the microneedles are planar microneedles, the STAR particles may have a substantially planar, i.e., flat, structure. The substantially planar, i.e., flat, STAR particles may have a height (thickness) of about 1 μm to about 1,000 μm, about 5 μm to about 500 μm, about 10 um to about 250 μm, about 50 μm to about 250 μm, about 50 μm to about 200 μm, about 50 μm to about 150 μm, about 75 μm to about 200 μm, about 75 μm to about 150 μm, about 75 μm to about 125 μm, or about 80 μm to about 120 μm. In some embodiments, the height of the microneedles is consistent throughout the length of the microneedle. That is, the height of the microneedle is the same where the microneedle contacts the core structure as at the tip. In some embodiments, the height of the microneedle particles decreases along the length of the microneedle. The height of the microneedle may be largest where the microneedle particle contacts the core structure and smallest at the tip.

In some preferred embodiments of tapered STAR particles, the height of the core in the center of the STAR particle is from 100 μm to 150 μm, and the radius of curvature at the tips of the microneedles of the STAR particle is from 5 μm to 30 μm.

The STAR particles may be made of one or more biocompatible materials, such as metals, polymers, biopolymers, ceramics, bioactive agents, sugars, sugar alcohols, or a combination thereof. The bioactive agents generally may include one or more drugs, one or more sensors, one or more cosmeceuticals, or a combination thereof. Therefore, the STAR particles may be made of a combination of bioactive components (drugs, small molecule excipients (e.g., trehalose), sensors, cosmeceuticals, or a combination thereof) and inactive components (metals, polymers, ceramics, sugars, etc.). If a portion of the STAR particle remains in and/or on a biological tissue after removal of the STAR particle, then the portion of the STAR particle remaining in and/or on the biological tissue may include at least one bioactive component, at least one inactive component, or a combination thereof.

In some embodiments, the STAR particles are made of water-insoluble material(s). In some embodiments, the STAR particles are made of, or include, at least one water-soluble and/or erodible material. When the STAR particles are made of water-soluble and/or erodible material(s), the STAR particles or a portion thereof may safely degrade if left in a biological tissue, or after disposal. In one example, the STAR particle has a matrix structure, which may consist of or include a water-soluble or bioerodible material. As used herein, the term “bioerodible” means that the structure/material degrades in vivo or ex vivo by dissolution, enzymatic degradation, hydrolysis, erosion, resorption, chemical reaction or a combination thereof. It would be understood that “ex vivo” refers in this case to STAR particles on the tissue surface, or otherwise present in the environment but not necessarily in contact with a biological tissue. Other methods of degradation of water-soluble and/or water insoluble STAR particles include, but are not limited to, dissolution, hydrolysis, degradation upon contact with sunlight (i.e., UV rays), or degradation resulting from a reaction with environmental factors (e.g., oxygen).

In some embodiments, the STAR particle is a metal microneedle particle. A metal microneedle particle is one in which all or substantially all of the structure of the microneedle particle is made of a metal or metal alloy (e.g., a stainless steel). In some other embodiments, a majority of the STAR particle is made of such metal or metal alloy materials.

In some embodiments, the STAR particle is a polymeric microneedle particle. A polymeric microneedle particle is one in which all or substantially all of the structure of the microneedle particle is made of one or more polymeric materials (e.g., biodegradable materials like poly(lactic-co-glycolic acid) (PLGA) or polycaprolactone (PCL) and/or water-soluble materials like carboxymethylcellulose or polyvinyl alcohol). In some other embodiments, a majority of the STAR particle is made of such one or more polymeric materials.

In some embodiments, the STAR particle is a ceramic microneedle particle. A ceramic microneedle particle is one in which all or substantially all of the structure of the microneedle particle is made of one or more ceramic materials (e.g., aluminum oxide, titanium dioxide, zinc oxide, iron oxides). In some other embodiments, a majority of the STAR particle is made of such one or more ceramic materials.

In some embodiments, all or substantially all of the structure of the microneedle particle is made of a bioactive agent and/or another other SOI. In some embodiments, a majority of the STAR particle is made of one or more drugs.

In some embodiments, the STAR particle is an excipient microneedle particle. An excipient microneedle particle is one in which all or substantially all of the structure of the microneedle particle is made of one or more pharmaceutically acceptable excipient materials known in the art (e.g., sugar, salt, starch, etc.).

In some embodiments, the STAR particle has a structure that is formed of a combination of (i) at least one metal (or metal alloy), (ii) at least one polymeric material, (iii) at least one ceramic material, and/or (iv) a at least one bioactive component.

The STAR particles provided herein may be made by any suitable method capable of forming a desired geometric shape of the STAR particles. Non-limiting examples of such methods include molding, mechanical or chemical etching, laser cutting, 3D printing, or other microfabrication techniques known in the art. For example, the STAR particles may be formed by laser etching a sheet of a material. As a further example, the STAR particles may be made using a molding process that may include placing a material of construction in a mold having cavities that correspond to the desired geometry of the resulting STAR particles. The material of construction may be a polymer or precursor thereof, and may be loaded into the mold in a powder or liquid form (e.g., molten polymer and/or polymer dissolved or dispersed in a vehicle), and then solidified into solid monolithic form in the mold. In another example, an array of discrete particles is formed from a solid sheet of the material by a process that includes at least one of etching, punching, or cutting, such as laser cutting. The STAR particles also may be sintered, densified, and/or mechanically hardened via heating, cooling, chemical modification, light exposure, drying, compressing, and/or other processes.

### STAR Particle-Containing Compositions

In various embodiments, the STAR particles are provided as a composition that facilitates application of the STAR particles to a target tissue site, e.g., a biological tissue surface, such as mammalian skin. For example, the composition may include or consist of STAR particles dispersed in a suitable medium that can flow. The medium may be a liquid, solution, lotion, cream, ointment, gel, paste, emulsion, aerosol foam or spray, powder, or semi-solid. The suitable medium is referred to herein as a “vehicle”.

Essentially any suitable biocompatible vehicle may be used in the STAR particle-containing composition. The vehicle may be an aqueous medium and/or a non-aqueous medium. The vehicle may include water, stabilizers, pH modifiers, thickening agents, or other pharmaceutically acceptable excipients known in the art for use in topical therapeutic applications, including materials that listed as Generally Recognized as Safe (GRAS) by the U.S. Food and Drug Administration.

The STAR particle-containing composition may include one or more bioactive agents (e.g., a therapeutic or prophylactic agent) and/or other substances of interest (e.g., diagnostic agents, sensors, cosmetics/cosmeceuticals). The bioactive agent, and/or the other SOI, may be disposed in or on the STAR particles, in the vehicle, or both in or on the STAR particles and in the vehicle. In some embodiments, the bioactive agent is dissolved in the vehicle. In some embodiments, the bioactive agent is dispersed, i.e., as a particulate suspension, in the vehicle.

The STAR particle-containing composition generally has a viscosity suitable for its intended storage, packaging, and use (e.g., application to a target tissue). In some embodiments, the STAR particle-containing composition is a viscous composition, having a viscosity of at least 1,000 cP. In some embodiments, the composition has a viscosity of about 1,000 cP to about 200,000 cP, about 1,000 cP to about 150,000 cP, about 1,000 cP to about 100,000 cP, about 1,000 cP to about 75,000 cP, or about 1,000 cP to about 50,000 cP. In some embodiments, the STAR particle-containing composition is a non-viscous composition, having a viscosity of less than 1,000 cP, for example, about 5 cP to about 500 cP, about 5 cP to about 250 cP, or about 5 cP to about 100 cP. In some embodiments, the STAR particle-containing composition has a viscosity of about 1 cP.

The concentration of STAR particles in the vehicle may be selected based on the particular application, but generally would be selected to achieve the intended function of the STAR particles at a particular tissue site. For example, the STAR particle concentration may be selected to be sufficient to create enough holes in the stratum corneum to deliver a therapeutically effective amount of a bioactive agent into the skin at the site of application of the STAR particle-containing composition.

In some embodiments, the concentration of STAR particles in the vehicle ranges from about 100 to about 100,000 particles per cm³ of the vehicle. In some embodiments, the concentration of STAR particles in the vehicle ranges from about 500 to about 50,000 particles per cm³ of the vehicle. In some embodiments, the concentration of STAR particles in the vehicle ranges from about 1,000 to about 25,000 particles per cm³ of the vehicle. In some embodiments, the concentration of STAR particles in the vehicle is greater than 10,000 particles per cm³ of the vehicle. In some embodiments, the concentration of STAR particles in the vehicle is less than 10,000 particles per cm³ of the vehicle.

In some embodiments, the concentration of STAR particles in the vehicle ranges from about 0.1 wt % to about 30 wt % of the vehicle. In some embodiments, the concentration of STAR particles in the vehicle ranges from about 1 wt % to about 20 wt % of the vehicle. In some embodiments, the concentration of STAR particles in the vehicle ranges from about 5 wt % to about 15 wt % of the vehicle. In some embodiments, the concentration of STAR particles in the vehicle ranges from about 8 wt % to about 12 wt % of the vehicle. In some embodiments, the concentration of STAR particles in the vehicle is about 5 wt % to about 10 wt % of the vehicle.

STAR particle compositions may also include at least one SOI. As used herein, “substance of interest” or “SOI” refers to a molecule or collection of matter that has a prophylactic, therapeutic, diagnostic, or cosmetic purpose. Substances of interest may include, but are not limited to, active pharmaceutical ingredients, vaccines, allergens, vitamins, cosmetic agents, cosmeceuticals, diagnostic agents, sensors, markers (e.g., colored dyes or radiological dyes or markers), other bioactive agents, and other materials that are desirable to introduce into or onto a biological tissue. A list of substances of interest is included in U.S. Pat. No. 11,291,816, which is incorporated herein by reference.

The SOI may be a small molecule, polymer, peptide, or biologic agent. In some embodiments, the SOI is a biological agent or a living organism. In further embodiments, the SOI has electronic properties. For example, the SOI may be responsive to radio-frequency identification (RFID).

### Methods of Incorporating a Substance of Interest

Incorporating a substance (or substances) of interest into and/or onto STAR particles may enhance the SOI's intended effect, alter the bioavailability and/or biodistribution of the SOI, or endow the SOI with new properties the SOI does not possess on its own. As used herein, “SOI” refers to a molecule or collection of matter that has a prophylactic, therapeutic, diagnostic, or cosmetic purpose. Substances of interest may include, but are not limited to, active pharmaceutical ingredients, vaccines, allergens, vitamins, minerals, cosmetic agents, cosmeceuticals, nutraceuticals, diagnostic agents, sensors, markers (e.g., colored dyes or radiological dyes or markers), fragrances, other bioactive agents, and other materials that are desirable to introduce into or onto a biological tissue. A more extensive list of substances of interest is included in U.S. Pat. No. 11,291,816, which is incorporated herein by reference.

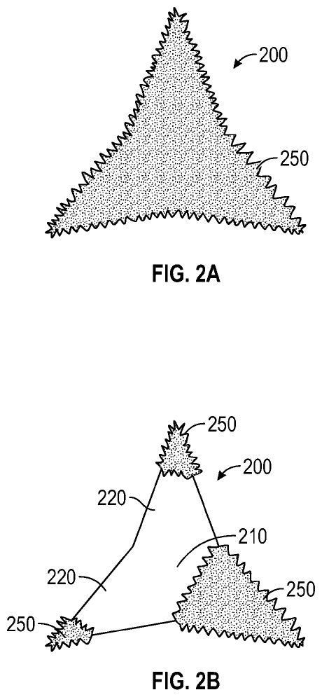

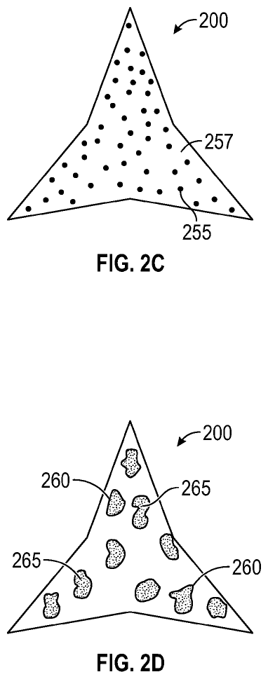

FIGS. 2A-2D depict embodiments of a STAR particle 200 having a SOI incorporated therewith. Referring to FIGS. 2A-2B, the STAR particles 200 have a coating 250, where the coating includes a SOI. In some embodiments, as shown in FIG. 2A, the coating 250 may cover the entire surface area of the STAR particle 200. In some embodiments, as shown in FIG. 2B, the coating 250 may cover only a portion (or portions) of the STAR particle 200. For example, the coating 250 may be applied to at least a portion of the microneedles 220, the core structure 210, or a combination thereof. In some embodiments, the coating (comprising one or more substances of interest) may be applied to the core structure only, only to one or more of the microneedles, or only to the tip portions of one or more of the microneedles. Although the illustrated coating appears to have a jagged surface, it may be, and likely would be, smooth or at least less rough than illustrated.

In some embodiments, the STAR particle is coated with a single coating containing one or more substances of interest. In other embodiments, the STAR particle can be coated with two or more coatings, which may contain one or more substances of interest. A microneedle of a STAR particle may have the same or a different SOI (e.g., as a coating) from other microneedles of the STAR particle.

For example, a first coating containing a diagnostic SOI can be loaded onto a first microneedle of a STAR particle, a second coating containing a second diagnostic SOI can be loaded onto a second microneedle of the STAR particle, and a third coating containing a third diagnostic sensor can be coated onto a third microneedle of the STAR particle. As a non-limiting example, the substances of interest in this embodiment may be ones configured to detect different infections, e.g., one microneedle each for a fungal, viral, and bacterial infections.

In some embodiments, the coating is a liquid, solution, emulsion, or suspension. The liquid, solution, or suspension may be a homogenous composition, and in some cases it may consist only of a SOI. Alternatively, the liquid, solution, emulsion, or suspension may be a heterogeneous mixture having at least one SOI and at least one carrier material. In some embodiments, the STAR particle, or a portion thereof, is submerged in the liquid, solution, emulsion, or suspension to form a coating. In some embodiments, the liquid, solution, emulsion, or suspension is sprayed, or otherwise deposited, onto the STAR particle, or a portion thereof, to form a coating. After being applied, the coating may remain in a liquid state, or it may dry to a solid or semi-solid form.

In some embodiments, the coating is a gas. Generally, the gas may be adsorbed and/or adsorbed into and/or onto the STAR particle, or a portion thereof, to form a coating. In one embodiment, the STAR particles are incubated with the gas, such that the gas is adsorbed into and/or onto the STAR particle, or a portion thereof. In another embodiment, the gas flows over a bed of STAR particles to the same effect.

In some embodiments, the coating is a solid. In some embodiments, the solid is deposited onto the STAR particle, or a portion thereof, in a manner that forms a coating. In some embodiments, the solid is co-formulated with a liquid or gas in a suspension, where the co-formulation may form a coating. The coating comprising a co-formulation may be applied to the STAR particles, or a portion thereof, according to the aforementioned methods pertaining to liquids. The liquid or gas component of the co-formulation may subsequently evaporate, or otherwise disappear, such that only the solid or semi-solid remains as a coating.

In some embodiments, the STAR particles are entirely or substantially constructed of a substance (or substances) of interest. That is, the STAR particles are constructed of a homogenous composition comprising the SOI. As used herein, “substantially constructed” means at least 60% by weight the SOI.

FIG. 2C depicts a STAR particle 200 containing a SOI 255, where the SOI is dispersed within a carrier material 257. Together, the SOI 255 and the carrier material 257 form a homogeneous or heterogeneous composition (i.e., a matrix). In some embodiments, the carrier material is a water soluble biocompatible material, a polymeric material (e.g., poly-lactic acid (PLA), poly-lactic-co-glycolic acid (PLGA) or polycaprolactone (PCL), polyvinyl alcohol (PVA)), such that the STAR particle is constructed of a polymeric matrix having the SOI dispersed within. In some embodiments, the carrier material is a water soluble material such as a sugar or carbohydrate (e.g., carboxymethylcellulose, trehalose, maltose). In some embodiments, the carrier material is a non-water soluble polymer. In other embodiments, the carrier material is made of any other substance that is generally regarded as safe, e.g., GRAS materials known in the art. The entire STAR particle, or a portion thereof, can be comprised of the matrix.

In some embodiments, the SOI may be incorporated into the STAR particle while it is synthesized. The SOI may be incorporated homogeneously or heterogeneously with the other materials or compounds required to synthesize the STAR particles 200. In some embodiments, STAR particles 200 may be synthesized in a single step. In some embodiments, STAR particles 200 are synthesized in a multi-step process. Some process steps may only involve the SOI, other steps may only involve materials that are not the SOI, such as excipients or fillers, and further steps may include both types of materials.

In some embodiments, the STAR particles are synthesized on or around a SOI. The SOI may be contained in a core or shell, such that the SOI is localized to the center of the STAR particle. In some embodiments, the SOI may be dispersed throughout the STAR particle. In other embodiments, the SOI can be localized to the tips of the microneedles on the STAR particle. For example, the SOI may be stacked or layered within the STAR particle. In further embodiments, the SOI may be loaded into the STAR particles after the STAR particles have been synthesized.

In some embodiments, the SOI undergoes a phase change during synthesis of the STAR particles. These phase changes may be triggered by several factors including, but not limited to, changes in temperature, pressure, or water and/or solvent content in, on, or around the STAR particles. The SOI may be applied as a gas, but changes phase to a liquid or a solid during the course of the STAR particle synthesis. Similarly, the SOI may be applied as a solid that changes phase to a liquid or a gas. The SOI may also be a liquid that changes phase to a gas or a solid.

In some other embodiments, the SOI is a solid particle and is applied as a dispersion in a liquid and/or gaseous carrier fluid, or is a solute dissolved in a liquid carrier, that leaves a solid deposit on the STAR particles after the carrier or solvent evaporates.

The SOI may also be a liquid or a gas. In some embodiments where the SOI is a liquid, the STAR particles 200, or portions thereof, may be submerged in the liquid SOI. The liquid of substance may also be sprayed, or otherwise deposited, onto the STAR particles 200. The STAR particles 200 may be configured to absorb the liquid SOI, thereby dispersing the SOI within the STAR particles 200.

In some embodiments, where the SOI is a gas, the gas generally may be absorbed and/or adsorbed into the STAR particles. The microneedles may be incubated with the gaseous SOI, or the gaseous SOI may be flowed over a bed of STAR particles 200. Absorption of the gaseous SOI is effective to disperse the SOI within the STAR particles. The gas may also be entrapped within the STAR particles during synthesis, effectively sealing or containing the gas within cavities of the STAR particle 200 when synthesis is complete.

FIG. 2D depicts a STAR particle 200 having a plurality of porosities 260 (i.e., voids or cavities). The porosities 260 may be uniformly distributed throughout the STAR particle. In some embodiments, a SOI 265 is deposited within the porosities 260. The SOI 265 may be an active agent alone, or in combination with a carrier material. The SOI 265 may be a liquid, gas, or solid, as described with respect to FIGS. 2A-2B. The porosities may be homogeneously distributed throughout the STAR particle or localized to certain regions of the STAR particle such as the core or on the microneedle tips. The pores may form a continuous or semi-continuous network throughout or substantially throughout the STAR particle. The porosities may, in part, provide shielding for the SOI such that the SOI has limited physical or chemical interaction outside of the intended use. In some embodiments, the shielding provided by the porosities may provide stabilization effects for the SOI.

In some embodiments where the SOI 265 is a liquid, the STAR particles 200, or portions thereof, may be submerged in the liquid SOI 265. The liquid of SOI 265 may also be sprayed, or otherwise deposited, onto the STAR particles 200. The liquid SOI 265 may settle into the porosities 260 of the STAR particle 200, where the SOI 265 may remain in a liquid state. The SOI 265 may also dry into a solid or semi-solid state.

In some embodiments, where the SOI is a gas, the gaseous SOI may be adsorbed onto the STAR particles, or a portion thereof. The STAR particles are incubated with the gaseous SOI, or the gaseous SOI may flow over a bed of STAR particles. Application of the gaseous SOI to the STAR particles may be effective to deposit the SOI within porosities of the STAR particles.

In some embodiments, where the SOI is a solid, the solid SOI may be deposited into porosities of the STAR particles. The solid SOI may also co-formulated with a liquid in a suspension, where the liquid suspension is applied to the STAR particles, settling within the porosities. The liquid may subsequently evaporate, such that only the solid SOI remains within the porosities.

In some embodiments, a composition is provided that includes a plurality of STAR particles configured to mechanically disrupt a biological tissue surface (e.g., stratum corneum), wherein the STAR particles have a heterogeneous structure. For example, the heterogeneous structure may include (i) a first SOI contained within a shell of carrier material, (ii) one or more layers of a first SOI stacked with one or more layers of a carrier material; or (iii) dispersed domains containing a first SOI, which may consist of a suspension of solid SOI-containing particles, dispersed in a solid medium of carrier material. In such embodiments, the carrier material may include or consist of one or more pharmaceutically acceptable excipients. The composition may further include a liquid vehicle in which the plurality of STAR particles are dispersed. The liquid vehicle may include a second SOI, for example, as a solute in the liquid vehicle, which may be an aqueous solution.

In some specific embodiments, a composition is provided that includes a plurality of STAR particles which include a first SOI which includes (i) a compound which absorbs UV radiation or otherwise is effective as a sunscreen, (ii) a perfume and/or (iii) an insect repellant. The composition may further include a liquid vehicle in which the plurality of STAR particles are dispersed. The composition may further include a second SOI on or in the STAR particles and/or in the liquid vehicle, for example, as a solute in the liquid vehicle, which may be an aqueous solution. The STAR particles may further include a carrier material, e.g., as a matrix material. The STAR particles may have a porous structure.

In some embodiments, the STAR particles are configured such that the first SOI is not released from the STAR particles following application of the composition to skin. It may be released later or not at all, depending for example on the particular function of the SOI. The first SOI, if released from the STAR particles may or may not be absorbed into the skin, depending for example, on the intended function of the first SOI. For example, a sunscreen, perfume or insect repellant may function without being absorbed into the skin. In some embodiments, the second SOI is configured to be absorbed into skin following application of the composition to the skin.

Any of the methods of incorporating a SOI with a STAR particle may be used alone or in combination. The methods may be used with a single SOI, or any number of substances of interest can be present in and/or on a single STAR particle.

### Methods of Delivering a Substance of Interest

Methods of delivering a SOI include contacting an area of skin, or another biological tissue surface, with a plurality of the STAR particles provided herein. Not wishing to be bound by any particular theory, it is believed that the methods of delivering a SOI disclosed herein may permit a SOI to be delivered to the biological tissue so that the SOI may have an increased effect.

In some embodiments, contacting a biological tissue surface or a region of skin with the plurality of the STAR particles comprises applying one or more forces to the STAR particles to ensure that at least a portion of the one or more microneedles at least partially penetrates the skin or other biological tissue at the intended site of application. The one or more forces may include a transverse force (e.g., perpendicular to the tissue surface), a shearing force (e.g., parallel to the tissue surface), or a combination thereof. For example, an intuitive rubbing motion may be applied to the STAR particles.

In some embodiments, at least a portion of a SOI may be released from a STAR particle before, while, and/or after the STAR particle has at least partially penetrated a biological tissue, while and/or after the STAR particle is actively applied to a biological tissue, while the STAR particle is in contact with a biological tissue, upon and/or after the removal of the STAR particle from the biological tissue, when a portion of the STAR particle remains on and/or in the biological tissue, or a combination thereof.

In some embodiments, at least a portion of a SOI may be released from a STAR particle by one or more mechanisms, including, but not limited to, diffusion through a portion of the STAR particle, dissolution into a biological tissue, mechanical separation from the STAR particle (e.g., peeling, breaking, or crumbling off), cleavage of a covalent and/or non-covalent bond (e.g., hydrolytic or enzymatic bond cleavage), cleavage of a physicochemical force (e.g., change in electrostatic interactions due to pH change), swelling/deswelling of a material of which at least a portion of the STAR particle is formed (e.g., a gel), a phase change of a material of which at least a portion of the STAR particle is formed (e.g., melting, due, for example, to a temperature change), or a combination thereof. A SOI may be released from the STAR particles in a variety of ways. For example, the STAR particle, or a part thereof, may dissolve, disintegrate, degrade, or undergo a phase change.

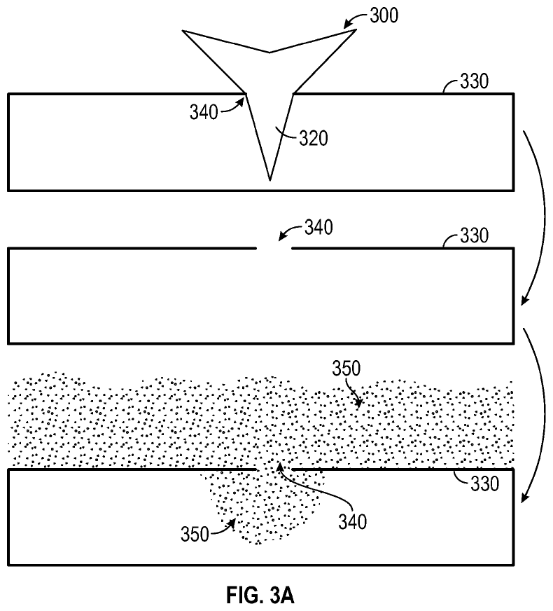

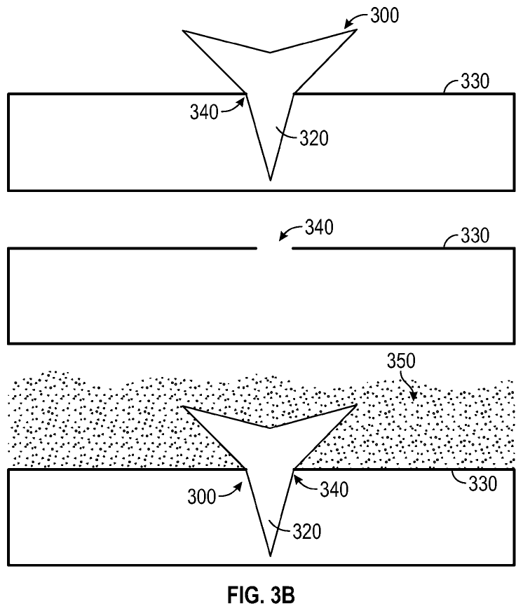

FIG. 3A depicts a method of delivering a SOI 350 where the tissue 330 is pre-treated with STAR particles 300 so that the SOI 350 is more effectively delivered. As used herein, “pre-treated” refers to applying STAR particles 300 to the biological tissue, absent a SOI 350. As shown in FIG. 3, pre-treating the tissue 330 with the STAR particles 300 forms a plurality of microchannels 340 in the biological tissue surface 330 (i.e., microchannels that originate at the outer surface of the biological tissue and penetrate deeper into the biological tissue below the outer surface). The microchannels 340 generally form when the one or more microneedles 320 at least partially penetrate a biological tissue 330.

The SOI 350 may then be delivered into a biological tissue 330 through a microchannel 340 created during pre-treatment with STAR particles 300. In some embodiments, a SOI 350 is contained within a vehicle applied to the tissue. In some embodiments, the SOI 350 is directly applied to the tissue 330. In some embodiments, the SOI 350 enters the microchannels 340 in the tissue 330 via diffusion. In some embodiments, the SOI 350 enters the microchannels 340 via convection or osmotic pressure. In some embodiments, there is an affinity-based interaction between the SOI 350 and a compound in the tissue 330, where the interaction draws the SOI 350 into the microchannels 340.

Delivery of the SOI into the pre-treated tissue may also be facilitate by application of an external stimulus. As used herein, “external stimulus” refers to any condition applied to the STAR particles, vehicle, SOI, and/or tissue. Forms of external stimulus may include, but are not limited to, exposure to visible light, changes in temperature, changes in pressure, addition, modification, or removal of chemical entities, application of ultrasound, application of electromagnetic radiation (e.g., ultraviolet, visible, or infrared radiation), and application of electrical or magnetic fields.

In some embodiments, the STAR particles push and/or pull the SOI into the microchannels, depositing the SOI within the tissue. This process may be accelerated by application of a mechanical force, convective force, and/or other external stimulus, to the tissue and STAR particles. Naturally occurring processes may also enhance the delivery of the SOI to the tissue. For example, where the tissue is the skin, a SOI may be solubilized by sweat or environmental moisture, enabling more effective delivery into the skin. The vehicle containing the SOI may also dry on the skin, which can improve or increase migration of the SOI into the microchannels. Alternatively, the SOI 350 precipitates into an active, particulate form when the vehicle dries.

FIG. 3B depicts application of a SOI 350 to anchor the STAR particles 300 to the tissue, for example where the STAR particles 300 are used to extract interstitial fluid or another substance through microchannel 340 for diagnostic purposes.

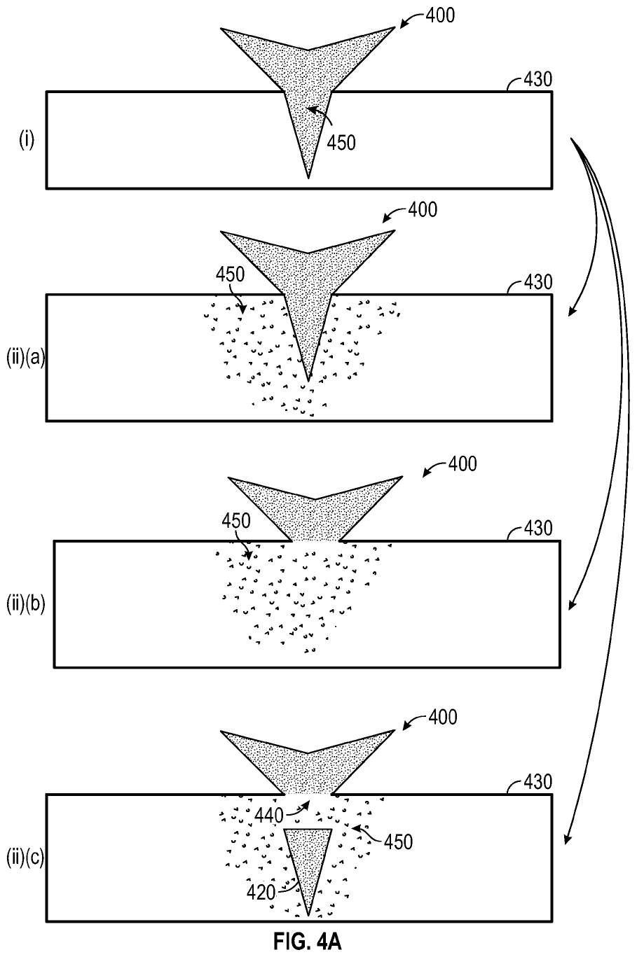

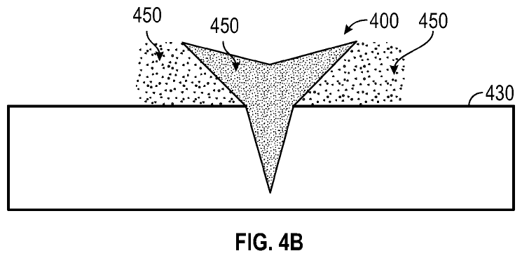

FIG. 4A-4B depicts a method of delivering a SOI 450 to tissue 430 from a STAR particle 400. As shown in step (i), STAR particles 400 containing a SOI 450, or coated in a SOI 450, may be applied to a biological tissue 430. The STAR particles 400 may be effective to mechanically disrupt the biological tissue 430, such that at least one microneedle 420 penetrates the tissue 430. In some embodiments, as shown in step (ii)(a), the SOI 450 may come off the penetrated microneedle 420, diffusing into the tissue 400.

In some embodiments, as shown in step (ii)(b), the microneedle penetrated in the tissue 430 may dissolve. As the microneedle dissolves, the SOI 450 may diffuse into the tissue. The dissolving microneedle may also create an osmotic gradient, facilitating release of the SOI 450 from the STAR particle 400. In some embodiments, as shown in step (ii)(c), the microneedle 420 penetrated in the tissue 430 may detach from the STAR particle 400 and remain within the tissue 430. The SOI 450 may diffuse from the detached microneedle 420 while the microneedle 420 remains within the tissue 430.

In some embodiments, the SOI 450 may come off the STAR particle 400 outside the tissue 430. For example, the SOI 430 may diffuse into the vehicle, and subsequently enter the skin through any microchannels 440 the STAR particles 400 have created. For example, in some embodiments, a method may include (i) wetting the skin surface with water or another aqueous liquid; (ii) applying/rubbing a dry formulation of STAR particles containing the SOI to the wetted skin surface, whereby the STAR particles, which are made of water soluble materials, dissolve in the water on the skin surface. This dissolution releases the SOI and makes the STAR particles disappear, dissolve or otherwise deactivate, and the SOI is then able to pass into/through micropores in the stratum corneum of the patient's skin. In these embodiments, the STAR particles are formulated so that they retain sharp tips for skin puncture during the initial rubbing (i.e., they do not dissolve before performing their intended purpose) but eventually dissolve to release the SOI and then become deactivated. Other embodiments may be envisioned in which the SOI is released from the STAR particles onto the skin surface followed by delivery of the SOI into/through the skin through the created microchannels.

FIG. 4B depicts a method by which the STAR particles 400 deliver a SOI 450 to the tissue surface. In some embodiments, the STAR particles 400 may adhere to the tissue 430, rather than or in addition to forming microchannels 440. Because the SOI 450 is in and/or on the STAR particles 400 themselves, the STAR particles 400 are effectively delivering and retaining the SOI 450 on the surface of the tissue 430.

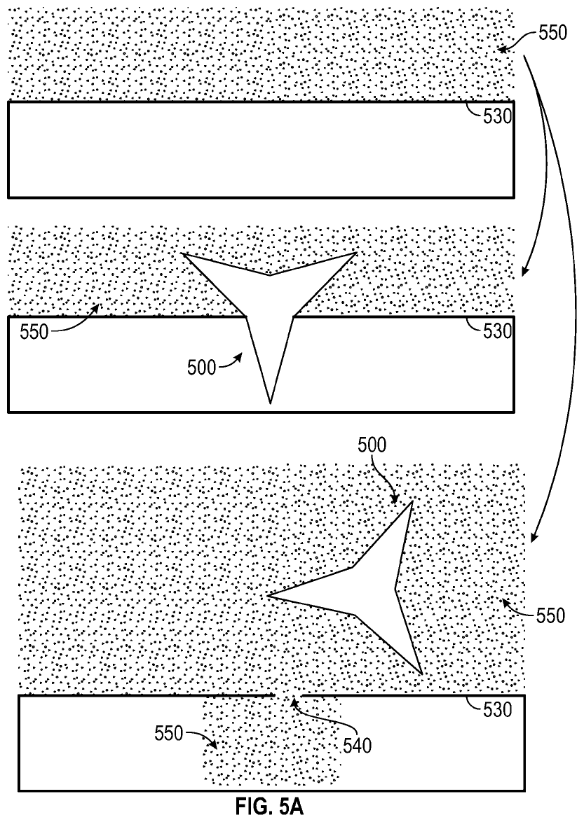

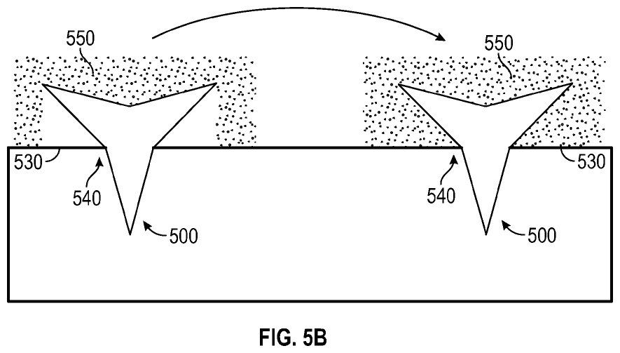

FIGS. 5A-5B depict methods of delivering a SOI 550 already present on or near the tissue surface 530. In some embodiments, the SOI 550 may be inherently present on the tissue surface 530. That is, the SOI 550 may be endogenously produced. For example, where the tissue 530 is skin, the SOI 550 may be sweat or other oils naturally produced by the body. In some embodiments, the SOI 550 may be environmental, such that the SOI contacts the tissue. For example, the SOI may be the ambient moisture in the air. In some embodiments, the SOI may be applied to the tissue, alone or dispersed within a vehicle. In another embodiment, the SOI 550 may be exogenously produced. For example, where the tissue is 530 is skin, the SOI 550 may be compounds produced by the skin's microbiome. As shown in FIG. 5A, a SOI 550 may be present on or near the tissue 530 regardless of whether STAR particles 500 are present.

Any of the aforementioned substances of interest 550 may be delivered into the tissue 530. As described with respect to FIG. 5, the STAR particles 500 create microchannels 540 in the tissue 530 through which the SOI 550 may enter the skin. The SOI 550 may enter the microchannels 540 by, for example, diffusion, convection, or osmosis. In some embodiments, an external force or stimulus may facilitate active delivery of the SOI 550 through the microchannels 540. Although not illustrated here, the STAR particle can remain in the skin and delivery of the SOI may still occur.

FIG. 5B depicts delivery of one of the aforementioned substances of interest 550 onto the tissue 530. In some embodiments, the STAR particles 500 adhere to the tissue 530, rather than or in addition to forming microchannels. The SOI 550 anchors to the STAR particles 500 adhered to the tissue 530, effectively delivering and retaining the SOI 550 on the surface of the tissue 530. The STAR particle may also adhere to appendages of the tissue. For example, the STAR particle may adhere to hair and enable localized delivery of a SOI to the hair shaft and/or follicle.

In some embodiments, a method is provided for transferring a biological substance from within a patient's skin to a surface of a patient's skin and/or into/onto a STAR particle, wherein the method includes contacting the skin with a plurality of STAR particles in a manner to create one or more holes in the stratum corneum of the patient's skin effective to cause the biological substance within the skin to be transferred onto the skin surface and/or into/onto the STAR particles. In some particular embodiments, the biological substance is collected from the skin surface and/or from the STAR particles for diagnostic and/or monitoring purposes. In some embodiments, the biological substance is interstitial fluid or a biomarker. The STAR particles may be configured to selectively uptake the biological substance. For example, the STAR particles may have an open porous structure and/or include a material which binds to a biomarker. In some other embodiments, the biological substance on the skin surface combines with a vehicle of a composition containing the STAR particles applied to the skin, e.g., it selectively binds with a component of the vehicle.

In some embodiments, a method of administering a SOI to a patient's skin is provided, wherein the method includes (i) contacting the patient's skin with a plurality of STAR particles in a manner to mechanically disrupt the stratum corneum of a patient's skin; (ii) transforming a composition comprising the STAR particles to form a film on the patient's skin; and (iii) incorporating the SOI into the film and/or into the interface between the skin and the film. The transforming may be or involve dissolution, melting, and/or plastic deformation of the STAR particles. The STAR particles may be a component of the film, but the film typically will include other components, which may be in the vehicle administered with the STAR particles and/or which may be a material added onto the skin after STAR particles have been applied to the skin.

In some embodiments, a method of administering a SOI to a patient's skin is provided, wherein the method includes (i) applying to the patient's skin a plurality of STAR particles; and (ii) rubbing the plurality of STAR particles in a manner effective to mechanically disrupt the stratum corneum of the patient's skin, wherein at least one SOI is released from the STAR particles during and/or after the rubbing. The SOI may be released from the STAR particles upon tip portions of microneedles of the STAR particles penetrating into the skin and dissolving or breaking off therein or on the surface of the skin. In some cases, the SOI is released from the STAR particles onto the surface of the skin but is not substantially absorbed into the skin during the rubbing. In some other cases, after the rubbing, the SOI is absorbed into the skin.

In some embodiments, a method is provided that includes (i) applying to a patient's skin a plurality of STAR particles comprising a SOI; and rubbing the plurality of STAR particles against the skin, wherein no or substantially no SOI is released from the STAR particles during the rubbing. In some cases, the SOI is released from the STAR particles onto the skin after the rubbing and then is absorbed into the skin. In some other cases, the SOI is released from the STAR particles after the rubbing and is not substantially absorbed into the skin. The SOI may include a compound which absorbs UV radiation or otherwise is effective as a sunscreen. The SOI may include a compound configured to evaporate from the skin surface, such as a perfume or insect repellant.

### Methods of Removing Biological Specimens

In some embodiments, the STAR particles 600 may remove a biological specimen 650 from within the tissue 630 or from the tissue surface 630. Exemplary biological specimens 650 include, but are not limited to, interstitial fluid, blood, sweat, sebum, biomarkers, cells, hair, tissue structures (e.g., collagen, elastin, other matrix structures), microbiota, pathogenic species such as bacteria, fungi or viruses, and/or other endogenous or exogenous specimens found within the skin or other bodily tissues. A biological specimen 650 may be removed from the tissue 630 for diagnostic, prognostic, and/or monitoring purposes. A biological specimen 650 may also be removed from the tissue 630 for prophylactic, therapeutic, and/or cosmetic applications. For example, pressure and/or fluid may be released from the skin, having a therapeutic effect (e.g., to reduce tissue swelling).

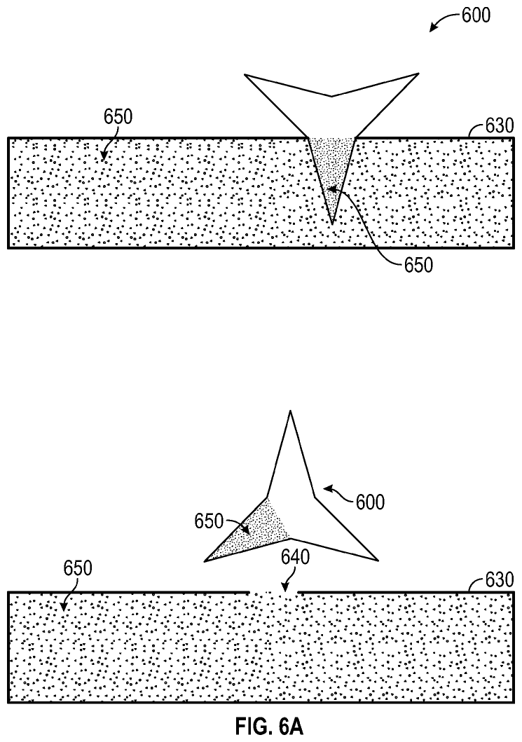

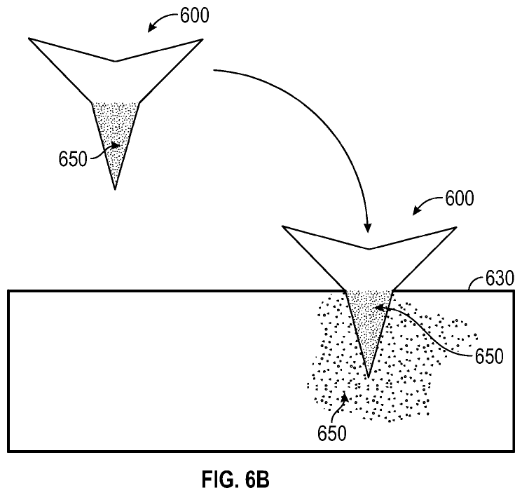

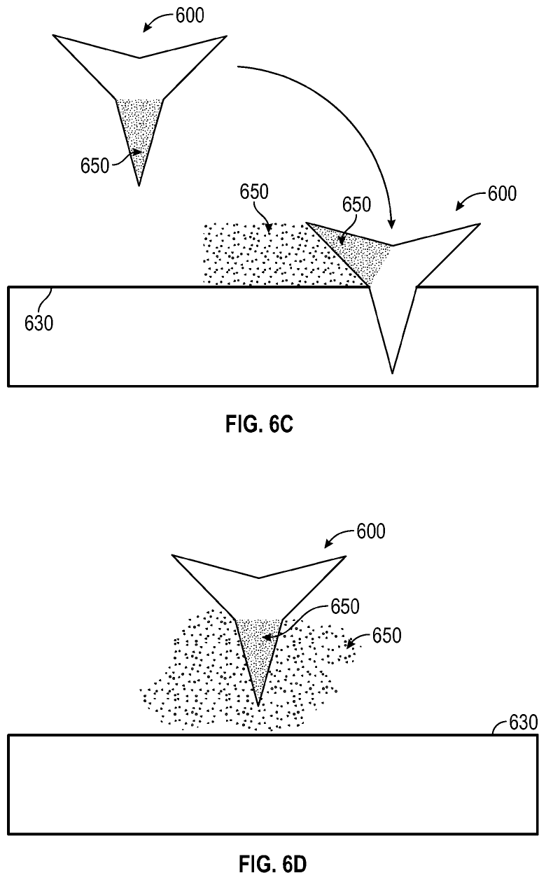

FIG. 6A depicts a method by which the biological specimen 650 is removed from the tissue. In some embodiments, the biological specimen 650 is collected with and retained on the STAR particle 600. For example, the STAR particle 600 may selectively uptake the biological specimen 650, mediated by binding, adsorption, or absorption of the biological specimen 650 with respect to the STAR particle 600. In some embodiments, the biological specimen 650 is removed from within the tissue 630. In some embodiments, the biological specimen 650 is removed from the tissue surface 630. In some embodiments, the biological specimen is cut away from the tissue 630 and extracted much like a tissue (e.g., skin) biopsy. In some embodiments, the STAR particle may be engineered to facilitate tissue 630 cutting and extraction such as by adding barbs, hooks, and/or shaving elements.

FIG. 6B depicts an embodiment where a biological specimen 650 carried on the surface and/or within a STAR particle 600 is released within a tissue 630, such as according to any of the methods described with respect to FIG. 4A.

FIG. 6C depicts an embodiment where the biological specimen 650 is redistributed to the tissue surface 630. The biological specimen 650 may be redistributed to the skin surface according to the methods described with respect to FIG. 4B.

FIG. 6D depicts an embodiment where the biological specimen 650 is introduced to, for example, a formulation on the tissue 630 or the surrounding environment. In some embodiments, the biological specimen 650 may be absorbed by the formulation or surrounding environment. In some embodiments, the biological specimen 650 may come off the STAR particle 600, such that it remains within the formulation or surrounding environment. In other embodiments, the biological specimen 650 evaporates into the surrounding environment.

In some embodiments, removal of the biological specimen 650 from the tissue 630 or tissue surface 630 may be facilitated by application of pressure to the tissue surface 630. For example, a positive pressure (e.g., squeezing) or a negative pressure (e.g., suction) may be applied to the tissue surface 630 to draw the biological specimen 650 out of the tissue 630 after the STAR particles 600 have been applied thereto. For example, the STAR particles are first applied to a patient's skin to create holes/pores in the stratum corneum, and then pressure or suction is applied to the skin in the area of the holes/pores to drive interstitial fluid containing biological materials to the skin surface for collection and analysis. For instance, the interstitial fluid can be collected and analyzed for one or more particular biomarkers or other biological materials that may be present therein.

### Methods of Making Star Particles Comprising a Substance of Interest

In some embodiments, a method of making STAR particles is provided that includes (i) dissolving or dispersing a SOI into a carrier material to form a fluid composition; and (ii) casting the fluid composition in molds to form the STAR particles, wherein the fluid composition comprises (i) a single phase, or (ii) two or more phases, such as a suspension. The carrier material in step (i) may be a molten material or dissolved in a volatile solvent, such that the casting in step (ii) may include freezing the molten material or evaporating the solvent, respectively.

In some embodiments, a method of making STAR particles is provided that includes (i) forming a plurality of porous STAR particles by any suitable means; and then (ii) contacting the formed porous STAR particles with a liquid, gas, solid, or any combination thereof, comprising a SOI in a manner such that the liquid, gas, solid, or a combination of two or three phases thereof is incorporated into pores of the STAR particles. The method may further include (iii) converting the SOI of the liquid incorporated into pores of the STAR particles into a solid or gaseous form, or converting the SOI of the gas incorporated into pores of the STAR particles into a solid or liquid form. The pores may be partially or completely filled.

In some embodiments, a method of making STAR particles is provided that includes (i) forming a plurality of STAR particles by any suitable means; and then (ii) adsorbing, absorbing, or spraying a solid form of a SOI into/onto the STAR particles. The STAR particles may be porous.

In some embodiments, a method of making STAR particles is provided that includes (i) providing a film which comprises a SOI and a carrier material; and (ii) cutting STAR particles out from the film by any suitable means, including but not limited to stamping or laser cutting. The carrier material may include one or more pharmaceutically acceptable excipients.

In any of the foregoing methods and compositions, the SOI may be or a bioactive agent, the STAR particles may be planar particles, and/or the STAR particles may be configured to mechanically disrupt the stratum corneum of a patient's skin, e.g., by rubbing the STAR particles thereon.

### Embodiments

Some embodiments of the present disclosure can be described in view of one or more of the following:
Embodiment 1. A composition comprising a plurality of STAR particles which are configured to mechanically disrupt a biological tissue surface, wherein the STAR particles comprise, at least in part, a porous structure.
Embodiment 2. A composition comprising a plurality of STAR particles which are configured to mechanically disrupt a biological tissue surface, wherein the STAR particles have pores comprising a first substance of interest (SOI) in (i) liquid or gaseous form contained in the pores, or (ii) solid form as a coating on surfaces defining the pores.
Embodiment 3. The composition of either of Embodiments 2, wherein the pores comprise the first SOI in the liquid form.
Embodiment 4. The composition of any one of Embodiments 2 to 3, wherein the pores comprise the first SOI in the solid form as a coating on surfaces defining the pores, optionally wherein the first SOI completely or substantially completely fills the pores.
Embodiment 5. The composition of any one of Embodiments 2 to 4, further comprising a vehicle in which the plurality of STAR particles are dispersed.
Embodiment 6. The composition of any one of Embodiments 2 to 5, wherein the vehicle comprises the first SOI and/or a second SOI.
Embodiment 7. The composition of any one of Embodiments 2 to 6, wherein the vehicle comprises no or substantially no SOI.
Embodiment 8. The composition of any one of Embodiments 2 to 7, wherein the first SOI comprises a bioactive agent.
Embodiment 9. The composition of any one of Embodiments 2 to 8, wherein the second SOI comprises a bioactive agent.
Embodiment 10. A composition comprising a plurality of STAR particles which are configured to mechanically disrupt a biological tissue surface, wherein the STAR particles comprise a gas.
Embodiment 11. The composition of Embodiment 10, wherein the gas is adsorbed or absorbed onto or into the STAR particles.
Embodiment 12. A composition comprising a plurality of STAR particles which are configured to mechanically disrupt a biological tissue surface, wherein the STAR particles have a heterogeneous structure comprising (i) a first substance of interest (SOI) contained within a shell of carrier material, (ii) one or more layers of a first SOI stacked with one or more layers of a carrier material; or (iii) dispersed domains containing a first SOI, which may consist of a suspension of solid SOI-containing particles, dispersed in a solid medium of carrier material.
Embodiment 13. The composition of Embodiment 12, wherein the carrier material comprises a pharmaceutically acceptable excipient.
Embodiment 14. The composition of either of Embodiments 12 or 13, further comprising a liquid vehicle in which the plurality of STAR particles are dispersed.
Embodiment 15. The composition of any one of Embodiments 12 to 14, wherein the vehicle comprises a second SOI.
Embodiment 16. The composition of any one of Embodiments 12 to 15, wherein the first and/or the second SOI comprises a bioactive agent.
Embodiment 17. The compositions of any one of Embodiments 1 to 16, wherein the STAR particles are planar particles.
Embodiment 18. The compositions of any one of Embodiments 1 to 17, wherein the STAR particles are configured to mechanically disrupt the stratum corneum of a patient's skin.
Embodiment 19. A method of making STAR particles, comprising dissolving or dispersing a substance of interest (SOI) into a carrier material to form a fluid composition, and casting the fluid composition in molds to form the STAR particles, wherein the fluid composition comprises (i) a single phase, or (ii) two or more phases, such as a suspension.
Embodiment 20. A method of making STAR particles, comprising forming a plurality of porous STAR particles, and then contacting the formed porous STAR particles with a liquid, gas, solid, or any combination thereof, comprising a substance of interest (SOI) in a manner such that the liquid, gas, solid, or a combination of two or three phases thereof is incorporated into pores of the STAR particles.
Embodiment 21. The method of claim 20, further comprising converting the SOI of the liquid incorporated into pores of the STAR particles into a solid or gaseous form, or converting the SOI of the gas incorporated into pores of the STAR particles into a solid or liquid form.
Embodiment 22. A method of making STAR particles, comprising forming a plurality of STAR particles, and then adsorbing, absorbing, or spraying a solid form of a substance of interest (SOI) into/onto the STAR particles.
Embodiment 23. The method of Embodiment 22, wherein the STAR particles are porous.
Embodiment 24. A method of making STAR particles, the method comprising providing a film which comprises a substance of interest (SOI) and a carrier material, and cutting one or more STAR particles out from the film.
Embodiment 25. The method of Embodiment 24, wherein the cutting comprises stamping or laser cutting.
Embodiment 26. The method of either of Embodiments 24 or 25, wherein the carrier material comprises one or more pharmaceutically acceptable excipients.
Embodiment 27. The method of any one of Embodiments 19 to 26, wherein the SOI comprises a bioactive agent.
Embodiment 28. The method of any one of Embodiments 19 to 27, wherein the STAR particles are planar particles.
Embodiment 29. The method of any one of Embodiments 19 to 28, wherein the STAR particles are configured to mechanically disrupt the stratum corneum of a patient's skin.
Embodiment 30. A method of transferring a biological substance from within a patient's skin to a surface of a patient's skin and/or into/onto a STAR particle, the method comprising contacting the skin with a plurality of STAR particles in a manner to create one or more holes in the stratum corneum of the patient's skin effective to cause the biological substance within the skin to be transferred onto the skin surface and/or into/onto the STAR particles.
Embodiment 31. The method of Embodiment 30, wherein the biological substance is collected from the skin surface and/or from the STAR particles for diagnostic and/or monitoring purposes.
Embodiment 32. The method of either of Embodiments 30 or 31, wherein the STAR particles are configured to selectively uptake the biological substance.
Embodiment 33. The method of any one of Embodiments 30 to 32, wherein the biological substance on the skin surface combines with a vehicle of a composition containing the STAR particles applied to the skin.
Embodiment 34. The method of any one of Embodiments 30 to 33, wherein the vehicle is configured to selectively uptake the biological substance.
Embodiment 35. The method of any one of Embodiments 30 to 34, wherein the biological substance is interstitial fluid or a biomarker.
Embodiment 36. The method of any one of Embodiments 30 to 35, further comprising applying pressure to the skin surface to withdraw the biological substance from the skin.
Embodiment 37. The method of any one of Embodiments 30 to 46, wherein the pressure is a positive pressure, such as squeezing the skin surface.
Embodiment 38. The method of any one of Embodiments 30 to 37, wherein the pressure is a negative pressure, such as applying suction to the skin surface.
Embodiment 39. A method of administering a substance of interest (SOI) to a patient's skin, the method comprising: contacting the patient's skin with a composition comprising a plurality of STAR particles and a vehicle in a manner to mechanically disrupt the stratum corneum of a patient's skin, transforming the composition to form a film on the patient's skin, and incorporating the SOI into the film and/or into an interface between the skin and the film.
Embodiment 40. The method of Embodiment 39, wherein the transforming comprises dissolution, melting, and/or plastic deformation.
Embodiment 41. A method of administering a substance of interest (SOI) to a patient's skin, the method comprising applying to the patient's skin a plurality of STAR particles, and rubbing the plurality of STAR particles in a manner effective to mechanically disrupt the stratum corneum of the patient's skin, wherein a SOI is released from the STAR particles during and/or after the rubbing.
Embodiment 42. The method of Embodiment 41, wherein the SOI is released from the STAR particles upon one or more tip portions of microneedles of the STAR particles penetrating into the skin and dissolving or breaking off therein or on the surface of the skin.
Embodiment 43. The method of either of Embodiments 41 or 42, wherein the SOI is released from the STAR particles onto the surface of the skin but not substantially into the skin during the rubbing.
Embodiment 44. The method of any one of Embodiments 41 to 43, wherein, after the rubbing, the SOI is absorbed into the skin.
Embodiment 45. A method comprising applying to a patient's skin a plurality of STAR particles comprising a substance of interest (SOI), and rubbing the plurality of STAR particles against the skin, wherein no or substantially no SOI is released from the STAR particles during the rubbing.
Embodiment 46. The method of Embodiment 45, wherein the SOI is released from the STAR particles onto the skin after the rubbing and then is absorbed into the skin.
Embodiment 47. The method of either of Embodiments 45 or 46, wherein the SOI is released from the STAR particles after the rubbing and is not substantially absorbed into the skin.
Embodiment 48. The method of any one of Embodiments 45 to 47, wherein the SOI comprises a compound which absorbs UV radiation or otherwise is effective as a sunscreen.
Embodiment 49. The method of any one of Embodiments 45 to 48, wherein the SOI comprises a compound configured to evaporate from the skin surface.
Embodiment 50. The method of any one of Embodiments 45 to 49, wherein the SOI comprises a perfume or insect repellent.
Embodiment 51. A composition comprising a plurality of STAR particles which comprise a first substance of interest (SOI) which comprises (i) a compound which absorbs UV radiation or otherwise is effective as a sunscreen, (ii) a perfume and/or (iii) an insect repellant.
Embodiment 52. The composition of Embodiment 51, wherein the STAR particles further comprise a carrier material.
Embodiment 53. The composition of either of Embodiments 51 or 52, wherein the STAR particles are configured such that the first SOI is not released from the STAR particles following application of the composition to skin.
Embodiment 54. The composition of any one of Embodiments 51 to 53, further comprising a liquid vehicle in which the STAR particles are dispersed.
Embodiment 55. The composition of any one of Embodiments 51 to 54, further comprising a second SOI on or in the STAR particles and/or in the liquid vehicle.
Embodiment 56. The composition of any one of Embodiments 51 to 55, wherein the second SOI is configured to be absorbed into skin following application to the composition to the skin.
Embodiment 57. The composition of any one of Embodiments 51 to 56, wherein the STAR particles are planar particles.
Embodiment 58. The composition of any one of Embodiments 51 to 57, wherein the STAR particles are configured to mechanically disrupt the stratum corneum of a patient's skin.

As used in this specification and the appended claims, the singular forms “a,” “an,” and “the” include plural referents unless the content clearly dictates otherwise. The term “about”, as used herein, indicates the value of a given quantity can include quantities ranging within 10% of the stated value, or optionally within 5% of the value, or in some embodiments within 1% of the value.

Modifications and variations of the methods and systems described herein will be obvious to those skilled in the art from the foregoing detailed description. Such modifications and variations are intended to come within the scope of the appended claims.

1. A composition comprising:
a plurality of STAR particles which are configured to mechanically disrupt a biological tissue surface,
wherein the STAR particles:
comprise, at least in part, a porous structure; or
have pores comprising a first substance of interest (SOI) in (i) liquid or gaseous form contained in the pores, or (ii) solid form as a coating on surfaces defining the pores.

2. (canceled)

3. The composition of claim 1, wherein the pores comprise the first SOI in the liquid form.

4. The composition of claim 3, wherein the pores comprise the first SOI in the solid form as a coating on surfaces defining the pores, optionally wherein the first SOI completely or substantially completely fills the pores.

5. The composition of claim 1, further comprising a vehicle in which the plurality of STAR particles are dispersed.

6. The composition of claim 5, wherein the vehicle comprises the first SOI and/or a second SOI.

7. The composition of claim 5, wherein the vehicle comprises no or substantially no SOI.

8. The composition of claim 1, wherein the first SOI comprises a bioactive agent.

9. The composition of claim 6, wherein the second SOI comprises a bioactive agent.

10-11. (canceled)

12. A composition comprising:
a plurality of STAR particles which are configured to mechanically disrupt a biological tissue surface,
wherein the STAR particles have a heterogeneous structure comprising (i) a first substance of interest (SOI) contained within a shell of carrier material, (ii) one or more layers of a first SOI stacked with one or more layers of a carrier material; or (iii) dispersed domains containing a first SOI, which optionally consists of a suspension of solid SOI-containing particles, dispersed in a solid medium of carrier material.

13. The composition of claim 12, wherein the carrier material comprises a pharmaceutically acceptable excipient.

14. The composition of claim 12, further comprising a liquid vehicle in which the plurality of STAR particles is dispersed.

15. The composition of claim 14, wherein the vehicle comprises a second SOI.

16. The composition of claim 12, wherein the first and/or the second SOI comprises a bioactive agent.

17. The compositions of claim 1, wherein the STAR particles are planar particles.

18. The compositions of claim 1, wherein the STAR particles are configured to mechanically disrupt the stratum corneum of a patient's skin.

19. (canceled)

20. A method of making STAR particles, comprising:
forming a plurality of porous STAR particles; and then
contacting the formed porous STAR particles with a liquid, gas, solid, or any combination thereof, comprising a substance of interest (SOI) in a manner such that the liquid, gas, solid, or a combination of two or three phases thereof is incorporated into pores of the STAR particles.

21. The method of claim 20, further comprising:
converting the SOI of the liquid incorporated into pores of the STAR particles into a solid or gaseous form, or
converting the SOI of the gas incorporated into pores of the STAR particles into a solid or liquid form.

22. A method of making STAR particles, comprising:
forming a plurality of STAR particles; and then
adsorbing, absorbing, or spraying a solid form of a substance of interest (SOI) into/onto the STAR particles.

23. The method of claim 22, wherein the STAR particles are porous.

24-50. (canceled)

51. A composition comprising:
a plurality of STAR particles which comprise a first substance of interest (SOI) which comprises (i) a compound which absorbs UV radiation or otherwise is effective as a sunscreen, (ii) a perfume and/or (iii) an insect repellant.

52-58. (canceled)
